(19) United States
(12) Patent Application Publication
(10) Pub. No.: US 2011/0178513 A1
(43) Pub. Date: Jul. 21, 2011

(54) METHOD AND DEVICE FOR INTERNAL TISSUE REMOVAL

(75) Inventor: Wolfgang Neuberger, Dubai (AE)

(73) Assignee: CreamOptee Industries Inc.

(21) Appl. No.: 13/010,217

(22) Filed: Jan. 20, 2011

Related U.S. Application Data
(60) Provisional application No. 61/297,114, filed on Jan. 21, 2010.

(51) Int. Cl. A61B 18/22

(52) U.S. Cl. 606/15; 606/14

(57) ABSTRACT

Methods and devices for internal tissue removal by means of laser energy are provided. In a preferred embodiment, a method for internal tissue removal in which tissue is removed in the form of fluid (liquid, vapor), solid debris and/or a combination of these is provided. Other embodiments disclose a method and a photodynamic method for the treatment of urological disorders such as benign prostatic hyperplasia. In another embodiment, a treatment system comprises infusion and vacuum hoses, a laser energy source with a wavelength or wavelength combinations for higher tissue ablation rates as well as reduced bleeding. As an advantageous feature, this invention can be applied to any kind of soft tissue which needs to be removed from the body. In addition, as it is not limited to hollow organs, it can be performed with or without using an endoscope. The method of internal tissue removal disclosed is safe, efficient, with enhanced outcomes and can be performed with less patient and surgeon stress.

### DOMESTIC PRIORITY UNDER 35 USC 119(E)

This application claims the benefit and priority of U.S. Provisional Application Ser. No. 61/297,114 filed Jan. 21, 2010, entitled “METHOD AND DEVICE FOR INTERNAL TISSUE REMOVAL” by Wolfgang Neuberger, which is incorporated by reference herein.

### BACKGROUND OF THE INVENTION

1. Field of the Invention

The present invention relates to the field of laser surgery and, in particular, relates systems and methods of applying laser radiation to treat undesired tissue within the body by removing it in the form of liquid, vapor or solid debris generated from application of laser energy.

2. Invention Disclosure Statement

There are many conditions in medicine which require tissue removal. Undesired tissue may include tumors and atheromatous plaques, excess fat in aesthetic treatments, or portions of prostate tissue in BPH cases, among others.

Tissue removal can be performed by means of different methods. Independently of the method used, main objective of this kind of treatment is the removal of the whole undesired tissue while preventing damage of surrounding tissue. In recent years, laser energy has been used in order to accomplish this aim.

Based on laser energy application on tissue, numerous approaches have been proposed. Laser techniques are usually preferred due to the laser's special capacity of delivering high amounts of power on reduced areas, thus improving treatment precision and accuracy and diminishing undesired effects on surrounding tissue.

Laser techniques can be broadly categorized in two main groups: coagulation and vaporization techniques. These approaches are founded on different concepts in order to achieve tissue removal.

Coagulation techniques are mainly based on destroying cells by the effect of the hyperthermia generated by the laser energy absorbed by tissues. These techniques are also sometimes called interstitial laser photocoagulation therapy. As a consequence of its principle of action, these techniques have been used to treat different kinds of tissues, namely, tumors, prostatic tissue (BPH), etc. As an example, in U.S. Patent Application No. 2006/0253178, Masotti discloses a device for treating tumors by means of interstitial laser thermotherapy. The device presented in the invention consists of a hollow needle which is inserted in tissue along with a light guide, and laser energy is applied to tissue in order to achieve tumor coagulation.

Coagulation techniques present various disadvantages, among them, the lack of immediate effects being the essential shortcoming. The desired effect of the therapy is not immediate and it often takes many weeks for the body to remove the dead cells. Inflammation is typical and can persist for extended periods of time.

As a consequence of the afore-mentioned drawbacks, tissue removal may be preferred in many cases over the more classical (interstitial) laser thermotherapy approach. Vaporization techniques consist of applying laser energy to tissue in order to vaporize it as required. Usually, this kind of technique is performed inside of hollow organs. For instance, U.S. Pat. No. 6,699,239 by Stiller discloses a laser instrument that can perform vaporization of biological tissue mainly in hollow organs. Laser instrument includes an optical waveguide used in combination with an endoscope. This invention is only useful for specific hollow organs and must be accompanied by an appropriate endoscope.

Fiedler et al., in U.S. Pat. No. 6,752,778, claims a device and method of suction removal of waste products such as smoke and tissue particles in the ablation of biological tissue by means of a laser beam, wherein the laser beam is directed to the tissue through the orifice of a tubular channel and the waste products are sucked through the orifice into the channel. This invention is mainly focused for treatments in which the waste products are smoke and small ablated tissue particles produced by laser energy such as photorefractive keratectomy.

In U.S. Patent Application No. 2008/0039828A1, Jimenez et al. teach a system and method for vaporizing tissue by means of a KTP laser, mainly for sterilization purposes. Target tissue is injected with a colorant in which KTP Laser radiation is highly absorbed. The usage of a KTP laser may be unsafe, as exposed in various articles. Furthermore, injecting a colorant may lead to allergic reactions and the procedure becomes more complex. Finally, laser energy causes tissue vaporization only, not being able to remove tissue in other forms (liquefied, for instance).

As another alternative, U.S. Pat. No. 7,367,969 by Stoltz et al. discloses a method of surgical material removal from a body by optical-ablation. Ablation from an outside surface of the body or inside the body is accomplished by controlling pulse energy from an amplifier, controlling the energy of a pulse and the pulse repetition rate, controlling the removal rate and the beginning and end of the ablation depending on the volume to be removed. No other means for improving optical-ablation apart from the mentioned above are disclosed.

With the aim of improving the surgical procedure of soft tissue removal by aspiration utilizing laser energy a device and method are disclosed by Zelickson et al. in the International Publication No. WO28073985A2. The laser energy is used for cleaving the soft tissue and coagulating small vessels. The simultaneous action of the laser energy and the reciprocating longitudinal motion of the laser soft tissue aspiration device allows the cleaved soft tissue to enter the soft tissue inlet port for tissue removal. In this device a coaxial fluid channel exists but differs from the present invention because the existence of this fluid system is only to provide fluid cooling of the laser energy transmission guide along its length.

Thus, due to the many drawbacks related to prior art method and devices used for tissue removal there is a need for a safe and efficient method of internal tissue removal such as the one disclosed in the present invention.

### OBJECTIVES AND BRIEF SUMMARY OF THE INVENTION

It is an objective of the present invention to provide an efficient method and device for internal tissue removal by means of laser energy.

It is another objective of the present invention to provide a treatment system for internal tissue removal using selected wavelength or wavelength combinations in order to achieve higher amounts of tissue ablation, reduced bleeding and enhanced tissue removal rates.

It is a further objective of the present invention to provide a method for internal tissue removal by means of laser energy in which tissue is removed in the form of fluid (liquid, vapor), solid debris and/or a combination of these.

It is yet another objective of the present invention to provide a method for internal tissue removal by means of laser energy for the treatment of urological disorders such as benign prostate enlargement in benign prostate hyperplasia (BPH); necrotic tissue; damaged tissue; infected tissue; unhealthy tissue; tumorous tissue; and unwanted adipose tissue.

It is still a further objective of the present invention to provide a photodynamic method for internal tissue removal by means of laser energy and an infusing fluid such as a photosensitizer whose absorption overlapped at least one wavelength output of the laser energy for the treatment of urological disorders such as benign prostatic hyperplasia.

Briefly stated, the present invention provides methods and device for internal tissue removal by means of laser energy. In a preferred embodiment, a method for internal tissue removal in which tissue is removed in the form of fluid (liquid, gases, vapor), solid debris or a combination of those is provided. Other embodiments disclose a method and a photodynamic method for the treatment of urological disorders such as benign prostatic hyperplasia. In another embodiment a treatment system comprises infusion and vacuum hoses, a laser energy source with one wavelength or wavelength combinations for higher tissue ablation rates as well as reduced bleeding. As an advantageous feature, this invention can be applied to any kind of soft tissue which needs to be removed from the body. In addition, as it is not limited to hollow organs, it can be performed with or without using an endoscope. The method of internal tissue removal disclosed is safe, efficient, with enhanced outcomes and can be performed with less patient and surgeon stress.

The above, and other objects, features and advantages of the present invention will become apparent from the following description read in conjunction with the accompanying drawings, (in which like reference numbers in different drawings designate the same elements).

### BRIEF DESCRIPTION OF FIGURES

FIG. 1 shows schematically an embodiment of a treatment system for internal tissue removal by means of laser energy.

FIG. 2 depicts an embodiment of a method for internal tissue removal by means of laser energy for the treatment of benign prostatic hyperplasia.

FIG. 3 depicts another embodiment of a system for use with a photodynamic method for internal tissue removal by means of laser energy and photosensitizer for the treatment of benign prostatic hyperplasia.

### DETAILED DESCRIPTION OF PREFERRED EMBODIMENTS

There are many disadvantages associated with the classical procedures used for tissue removal such as the interstitial thermotherapy approach for the treatment of benign prostate hyperplasia (BPH) or the treatment of tumors with interstitial coagulation. Essential shortcomings are the lack of immediate effects and the inflammation associated with the therapy that is usually painful condition and can persist for extended periods of time.

Advantageously, the safe and efficient method of internal tissue removal disclosed in the present invention overcomes the drawbacks presented in alternative therapies. Moreover, it provides a method of internal tissue removal by means of laser energy which is safe, efficient, with enhanced outcomes and that can be performed with less patient and surgeon stress. Waveguides such as optical fibers convey sufficient laser energy to the target tissue in order to achieve removal of substantial amounts of tissue in the form of fluid (liquid, vapor), gases, solid debris or a combination of these. At least one wavelength or wavelength combinations can be used in order to achieve higher amounts of tissue ablation as well as reduced bleeding. Furthermore, wavelength combinations can be chosen in order to achieve enhanced tissue removal rates thus diminishing treatment duration. As an advantageous feature, this method can be applied to any kind of soft tissue which needs to be removed from the body. In addition, as it is not limited to hollow organs, it can be performed with or without using an endoscope.

Preferred treatment systems for the enhanced removal of internal tissue comprises a laser energy source; spreading means to open an initial cavity in tissue to be treated; at least one waveguide to transmit laser energy into a tissue treatment site; means to introduce an infusing fluid to the treatment site such as an infusion pump; an infusing fluid which absorbs a wavelength emitted by said laser energy source; and means to remove debris and remnant infusing fluid during and/or after radiation treatment such as a suction pump. A waveguide to deliver laser energy to the treatment site includes, but it is not limited to, fiber optic based laser energy delivery devices.

Preferably, the treatment system further comprises means to place and verify position of distal ends of the spreading device, which is used to create an initial cavity, and the waveguide including, but not limited to, ultrasound devices, open magnetic resonance imaging devices (MRI) and/or endoscopes. Means to place and verify position of distal ends of both devices are required at the start and during the treatment in order to have control of the distal ends of the devices and diminish the possibility of damaging internal tissues. Additionally, spreading devices and waveguides can be introduced into the desired position with the aid of introducing means such as a hollow device or other introducing means known in the art. Preferably, spreading means is comprised of a shape-memory material, compressible for introduction into a hollow tube or other introducing means, which expands upon exiting a distal end of the hollow tube or introducing means to create an initial cavity for at least one waveguide and the infusing fluid to enter.

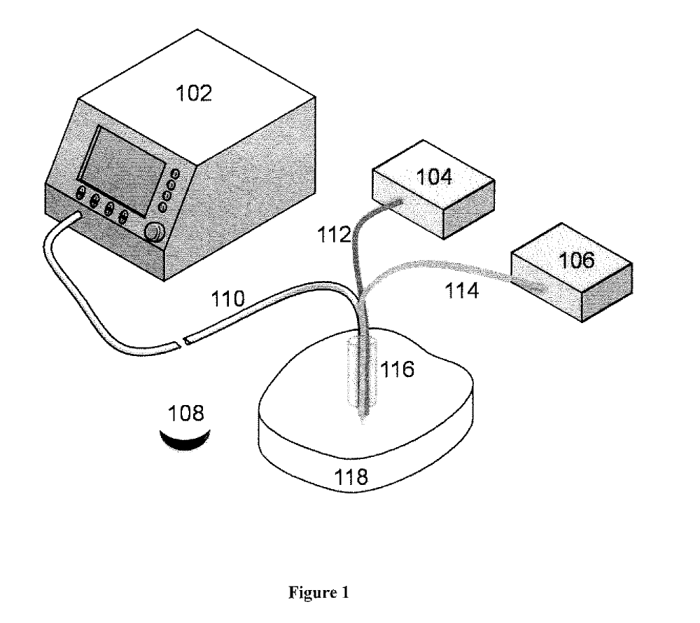

In a preferred embodiment depicted schematically in FIG. 1, a treatment system for enhanced internal tissue removal comprises laser energy source 102, infusion pump 104, suction pump 106 and spreading device 108. Laser energy is delivered with the aid of a single or a plurality of optical fibers 110. The tip of the optical fiber may be bent, twisted, may have a diffuser or may have any shape that ensures a proper and efficient delivery of laser energy to the target tissue. Infusion pump hose 112 connected to infusion pump 104 allows the introduction of spreading device 108 to create an initial cavity inside the region to be treated. Additionally, infusion pump hose 112 connected to infusion pump 104 allows the introduction of a solution that can absorb the emitted laser energy and/or may enhance tissue removal. Infusion pump hose 112 connected to infusion pump 104 also allows the introduction of filling material or healing substances or any other material that promotes the sealing, healing or filling of the opened cavity after tissue removal. Suction pump hose 114 connected to suction pump 106 allows the removal of the remnant infusing fluid or substances and freshly generated fluids, vapors, gases, solid debris and/or a combination of those, from the treatment site. Laser energy source 102 preferably delivers one or more laser wavelengths preselected from a range of 980 to 1940 nm in order to provide adequate tissue penetration and sufficient energy to heat water and blood of target tissue. Higher amounts of tissue ablation as well as reduced bleeding are, thus, achieved. Moreover, adjacent tissues are protected due to the initial cavity created which allows appropriate separation of the tissue to be treated from surrounding tissue. Depending on the penetrating fluid infused, one of the laser wavelengths may be selected according to its absorption properties. The infusing fluid overlaps at least one wavelength output of the laser energy source. Hollow tube 116 allows the insertion of optical fiber 110, infusion pump hose 112 and suction pump hose 114 in the appropriate position inside target tissue 118.

Other embodiments of the present invention include devices for delivering laser energy, infusing fluids and removing the penetrating fluid and generated fluids, vapors, solid debris and/or a combination of them given in other Patent Publication No. US 2006/0253112 A2 incorporated by reference herein.

Preferred methods of internal tissue removal based on laser energy comprise the steps of a) creating an initial cavity at a tissue treatment site; b) introducing an infusing fluid whose absorption overlapped at least one wavelength output of the laser energy; c) irradiating said tissue treatment site with adequate laser energy to remove desirable amount of unwanted tissue in the form of fluid (liquid, gas, vapor), solid debris and combinations of those; and e) removing debris and remnant infusing fluid from the treatment site during and after irradiation. In order to promote filling, healing or sealing of the treatment site, present invention methods of internal tissue removal based on laser energy further comprises an optional step of introducing healing or sealing substances inside the cavity created at the tissue treatment site. Many filling, healing and/or sealing substances are known such as growing factors, autografts, allografts, isografts, scaffolds, modified autografts, grafts including stem cells, and materials which support the sealing, filling or healing after tissue removal. These filling, healing or sealing substances can have a liquid, solid or gel form, but not limited thereof. Additionally, other substances can be added such as analgesics and anti-inflammatory drugs to enhance filling, healing or sealing processes. In order to irradiate the tissue treatment site the method further comprises the step of introducing at least one waveguide into the initial cavity.

In a preferred embodiment a method for enhanced internal tissue removal by means of laser energy comprises the steps of 1) selecting and/or marking the area to be treated; 2) making a percutaneous or surgical entry to introduce a hollow device in the area to be treated; 3) creating an initial cavity surrounding the tissue to be treated; 4) inserting a sterile optical fiber based laser energy delivery device until reaching the tissue to be treated; 5) introducing a penetrating fluid to the area to be treated; 6) irradiating the tissue with adequate laser energy; and 7) removing the remnant infusing fluid and generated fluids, vapors, gases, solid debris and/or a combination of those from the treatment site during and after irradiation.

In order to diminish any patient discomfort, anesthesia may be applied locally, close to an insertion site. Ultrasound, open MRI or an image scope e.g. endoscope may be used as guidance devices to place spreading devices and at least one optical fiber in the desired location and to confirm its position at the start and during the procedure. With the aim of enhancing tissue removal rates, more ablation in fibrotic tissue is obtained due to the augmented heating effect as a consequence of increased laser energy absorption by the infusing fluid. Though, the use of spreading devices and the creation of an initial cavity protect surrounding tissues from excessive high temperature while removing unwanted tissue. One or a combination of laser wavelengths is preferred in order to obtain high tissue ablation rates and reduced bleeding and/or selecting a laser wavelength according to the absorption properties of the infusing fluid. The infusing fluid is generally an aqueous solution, isotonic, hypertonic or hypotonic solution more preferably a saline solution, or may be any substance/solution that can absorb the emitted radiation, i.e. a preselected chromophore solution. The infusing fluid is introduced with the aid of an infusion pump connected with single or multiple hoses and can be at room temperature or at low temperature in order to be additionally used as a cooling fluid. The remnant infusing/penetrating fluid and generated fluids (vapors, gases, liquids), solid debris and/or a combination of these, are removed from the treatments site with the aid of a suction pump using single or multiple hoses.

Optionally, after tissue removal the initial cavity created to separate the unwanted tissue from surrounding tissue may be filled with a substance promoting the healing, filling or sealing of the treated area. Filling, sealing or healing materials may include growth factors, autografts, allografts, isografts, scaffolds, modified autografts, allografts or isografts including stem cells, or any other material that supports the sealing, filling or healing of the opened cavity after tissue removal.

One of the rising popular laser methods for treating BPH is the Laser-induced Interstitial Thermotherapy (LITT) in which a fiber is injected directly into the prostate and laser energy is applied from the inside to achieve tissue coagulation. Then, coagulated tissue is eliminated by the body. However, one of its main disadvantages is that it takes a long time to remove all the coagulated tissue from the body. Additionally, there is also inflammation associated with this procedure over extended periods of time, which leads to increased near-term patient discomfort. Another popular method for treating BPH consists of inserting an optical fiber through urethra to get close to the prostate and irradiating from there to ablate prostate tissue. Due to the nature of this procedure, the urethra may be damaged. Thus, there is a need for a fast and effective method of eliminating prostate tissue, while at the same time preserving the urethra.

In a preferred embodiment of the present invention, the method, used for enhanced internal tissue removal by means of laser energy delivery for the treatment of urological disorders such as benign prostate enlargement in benign prostate hyperplasia (BPH), comprises the steps of; 1) selecting and/or marking the area to be treated; 2) making a percutaneous or surgical entry to introduce a hollow device into the inside of the body tissue to be treated; 3) creating a newly passage surrounding the tissue to be removed with the aid of spreading means; 4) inserting a sterile optical fiber until reaching the tissue to be removed, preferably with a bent tip or side-fiber tip; 5) introducing an infusing solution to the treatment site; 6) applying sufficient quantities of laser energy with one or more preselected laser wavelengths; and 7) removing the remnant penetrating fluid and generated fluids, solid debris and/or a combination of these from the treatment site.

Anesthesia may be applied locally close to the insertion site. Spreading means comprise spreading devices or cape-like structures of a compressible shape-memory material, for introduction through a hollow device which expands upon exiting a distal end of said hollow device to create an initial cavity for at least one optical fiber, and infusing fluid to enter. The hollow device may be a needle type endoscope, a hollow tube, channel or any other similar hollow device.

When the shape-memory material comes out of the distal end of the hollow device it bends taking its pre-determined form due to its shape-memory property and it creates a new passage into the prostate lobe, for example, in a BPH procedure. Ultrasound, open MRI or endoscopes may be used as visual guidance to place the spreading devices or cape-like structures and at least one optical fiber's distal end in the desired location, and to verify their distal end position at the start and during the procedure. Laser energy delivery can be done in a “drill out” manner with a bent optical fiber. One or a combination of laser wavelengths preselected from a range of 980 to 1940 nm is preferred, e.g. about 980 nm, 1470 nm or 1900 nm, in order to obtain high tissue ablation rates and e.g. 980 nm, to get reduced bleeding. Nevertheless, at least one of the wavelength outputs of the laser energy source overlaps the absorption properties of the penetrating (infusing) fluid. The infusing fluid is generally an aqueous solution, more preferably a saline solution, where 980±30 nm, 1470±60 nm, or 1900±60 nm are particularly useful, or may be any substance that can absorb the emitted radiation i.e. a preselected chromophore in a solution. The infusing fluid is introduced with the aid of an infusion pump using single or multiple hoses. The remnant penetrating fluid and generated fluids, solid debris and/or a combination of these are removed from the treatment site with the aid of a suction pump using single or multiple hoses. Optionally, after tissue and waste removal, healing or sealing substances may be introduced inside the cavity to promote enhanced healing or sealing of the treated area. As an example, a combination of growing factors and medicines may be administered to enhance healing process. Then, the spreading devices or cape-like structures are removed with the aid of the hollow device used for insertion.

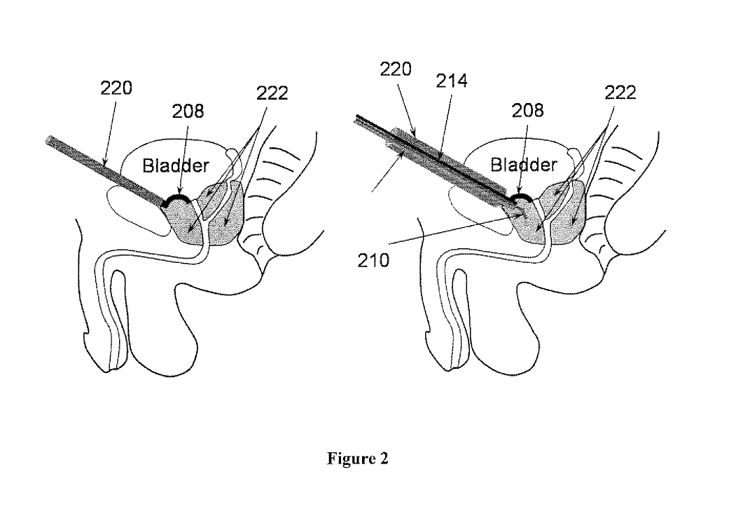

FIG. 2 illustrates a method for enhanced internal tissue removal by means of laser energy delivery for BPH treatment. Once the target tissue is properly located, needle type endoscope 220 is introduced into the inside of the body tissue to be treated. Through needle type endoscope 220 an initial cavity surrounding the unwanted tissue is created by introducing spreading device 208 of shape-memory material. When spreading device 208 comes out of the distal end of needle type endoscope 220 it bends taking its pre-determined form due to its shape-memory property. With spreading device 208 in the appropriate position at least one optical fiber 210 is inserted through needle type endoscope 220. Optical fiber 210 may have a bent tip to ensure enhanced irradiation and removal of the unwanted tissue. Ultrasound, open MRI or endoscopes may be used as guidance devices to place spreading device 208 and at least one optical fiber 210 in the desired location and to confirm their positions during the procedure. An infusing fluid to enhance tissue removal is introduced through infusion pump hose 214 into enlarged prostate 222 while laser energy is delivered through optical fiber 210. The penetrating fluid and generated fluids, solid debris and/or a combination of these are removed from enlarged prostate 222 through suction pump hose 214 with the aid of a suction pump. After complete removal of unwanted tissue, excessive fluids and/or solid debris, a solution combining growth factors and healing medicines is infused through infusion pump hose 212 into enlarged prostate 222 to enhance healing the treated area and/or sealing of cavity initially formed. Spreading device 208 may be removed from the treatment site before or after infusing healing and/or sealing substances with the aid of needle type endoscope 220 used for insertion.

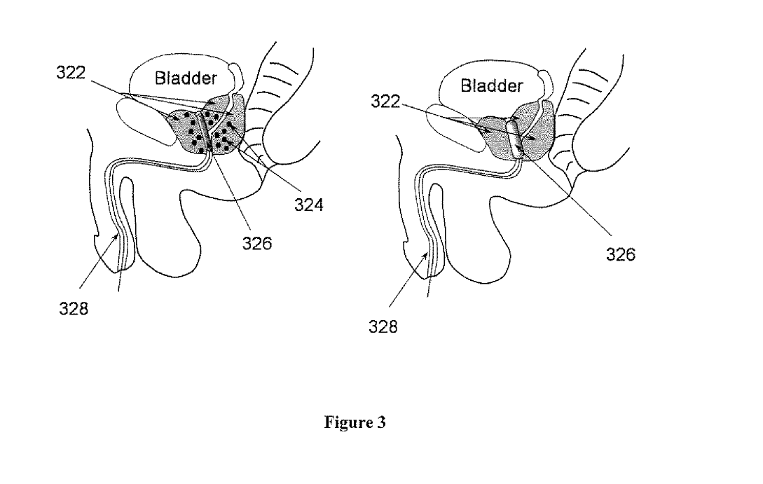

In another embodiment a photodynamic method for enhanced internal tissue removal by means of laser energy and photosensitizing agent for the treatment of urological disorders such as benign prostate enlargement in benign prostatic hyperplasia is depicted in FIG. 3. Photosensitizing agent 324 that can absorb the emitted radiation usually used in PDT treatments is applied by means of balloon 326, letting enlarged prostate 322 capture photosensitizing agent 324 and then irradiating through diffusing balloon 326 from urethra 328. Instead of using a delivery device, the photosensitizing agent may be injected directly into the unwanted tissue with the aid of a needle-type device or the like. After appropriate time the photosensitizing agent is distributed inside the target tissue and subsequently laser energy is applied with the aid of a diffusing balloon inserted through the urethra. Photodynamic therapy is a minimally-invasive therapy based on the administration of a photosensitizing agent which preferentially accumulates in hyperplasic tissue, so that after tissue accumulation in an oxygenated environment it is activated by light of a specific wavelength. The combination of these three elements, photosensitizing agent, light and oxygen, produces a cytotoxic effect due to the excited state-reactive singlet oxygen produced. Because PDT cytotoxic effect is based on a photochemical reaction there is little or no tissue heating thus the urethra is preserved and virtually no damage occurs due to heating effects. In contrast with most other PDT approaches which rely on ambient oxygen species in and around a treatment site, in order to enhance PDT effect here, oxygen may be administered to the target tissue with the aid of an infusion pump to increase the oxygen content in the area to be treated.

The present invention is further illustrated by the following examples, but is not limited thereby.

### Example 1

Present example refers to the treatment of benign prostate enlargement in BPH. The doctor first identifies the area to be treated and the amount and location of the unwanted tissue to be removed. Under ultrasound guidance, a hollow needle is introduced into the hyperplasic lobe. Before needle introduction, anesthesia is applied locally, close to insertion site. Then, a spreading device is introduced through hollow needle. Under ultrasound guidance, the spreading device protrudes from the distal tip of the hollow needle and creates an initial cavity which separates the tissue to be treated from surrounding tissue. Next, two sterile optical fibers with two cannulae attached are inserted through the hollow needle into the area to be treated, protruding from the distal end of the hollow needle inside the cavity previously created with spreading device. Once the optical fibers are in place, saline solution is infused through one of the cannula attached to the optical fibers with the aid of an infusion pump. While filling the area to be treated, laser energy of 1470 nm and 980 nm are delivered in alternate sequence through each optical fiber. This laser wavelength combination allows higher amounts of tissue ablation due to the augmented heating effect as a consequence of increased laser energy absorption by the saline solution as well as reduced bleeding. On the other hand, spreading device inside cavity prevents or diminishes damaging surrounding tissue while desired hyperthermia is achieved. During irradiation, remnant saline solution and generated fluids and solid debris are removed from the treatment site through the remaining cannula attached to the optical fibers with the aid of a suction pump. Next, an appropriate amount of solution containing growth factors, an anti-inflammatory drug and an analgesic is administered through the cannula previously used for infusing saline solution. Spreading device is removed through the remaining cannula attached to the optical fiber.

### Example 2

Present invention can also be used to remove patient's dead, damaged or infected tissue to improve the healing potential of remaining healthy tissue. This example refers to the removal of unwanted necrotic tissue such as necrotizing fasciitis. The doctor first identifies the area to be treated and the amount and location of the unwanted tissue to be removed. Under ultrasound guidance, a hollow needle is introduced into the area to be treated. To avoid patient discomfort, anesthesia is applied locally before needle insertion. Through the hollow needle a spreading device of compressible shape-memory material is introduced, which expands upon exiting the distal end of the needle creating an initial cavity surrounding the tissue to be treated. Correct positioning is achieved under ultrasound guidance as well. Then, a sterile optical fiber with a cannula attached is inserted through the hollow needle into the area to be treated, protruding from the distal end of the hollow needle in the passage, newly created by the spreading device. Once the optical fiber is in place, saline solution is infused through the cannula with the aid of an infusion pump, filling the created initial cavity. Once the initial cavity is filled with sufficient amount of saline solution, laser energy of 1470 nm is delivered through the optical fiber. Due to the augmented heating effect as a consequence of increased laser energy absorption by the saline solution, ablation rate in necrotic tissue is enhanced. After irradiation, remnant saline solution and generated fluids and solid debris are removed from the treatment site through the cannula attached to the optical fiber with the aid of a suction pump. Then, the spreading device can be also sucked through the cannula with the aid of the suction pump or, after removing the optical fiber and the cannula, it can be sucked through the hollow needle.

### Example 3

Present example illustrates how to remove the increased abnormal tissue of benign prostatic hyperplasia with the photodynamic method of this invention. The urologist first identifies the area to be treated and the amount and location of the unwanted tissue to be removed. With the aid of an endoscope an optical fiber with a cannula attached is introduced through urethra up to the treatment site. The tip of the optical fiber has a diffuser to ensure a proper and efficient delivery of laser energy to the enlarged prostatic lobe. The diffuser tip is surrounded by a balloon covered with the photosensitizing agent. Once in place, the balloon is inflated allowing the proper positioning of the diffuser inside the urethra and the delivery of the photosensitizing agent to the prostatic lobe through the urethra walls. After appropriate time the photosensitizing agent is distributed inside the enlarged prostatic tissue and subsequently laser energy absorbed by the photosensitizing agent is applied through the diffuser. While irradiating, oxygen is administered to the prostatic lobe through the cannula with the aid of an infusion pump to increase the oxygen content in the area to be treated and to enhance the PDT effect. After irradiation, generated fluids and solid debris are removed from the treatment site through the cannula attached to the optical fiber with the aid of a suction pump.

### Example 4

In order to remove increased abnormal tissue of benign prostatic hyperplasia an enhanced photodynamic method is used. The urologist first identifies the area to be treated and the amount and location of the unwanted tissue to be removed. Under ultrasound guidance, a hollow needle is introduced into the hyperplasic lobe and a photosensitizing agent is delivered. Before needle introduction, anesthesia is applied locally, close to insertion site. After an appropriate period of time the photosensitizing agent is distributed inside the enlarged prostatic tissue. Again, a hollow needle is introduced into the hyperplasic lobe creating an initial cavity surrounding the tissue to be treated. Then, a sterile optical fiber with a cannula attached is inserted through the hollow needle into the area to be treated, protruding from the distal end of the hollow needle. Once the optical fiber is in place, oxygen is infused through one of the cannula attached to the optical fiber with the aid of an infusion pump. While infusing oxygen, laser energy absorbed by the photosensitizing agent is delivered through the optical fiber. After irradiation, generated fluids and solid debris are removed from the treatment site through the cannula attached to the optical fiber with the aid of a suction pump.

### Example 5

Present invention can also be used to remove unwanted adipose tissues. A professional first identifies the area to be treated and the amount and location of the unwanted tissue to be removed. Under ultrasound guidance, a hollow device such as a stainless-steel tube is introduced into the area to be treated. Before introducing the stainless-steel tube into the deep fat layer, anesthesia is applied locally, close to insertion site. Then, a spreading device is introduced through stainless-steel tube and under ultrasound guidance it is positioned between the deep fat layer and the superficial fat layer, creating an initial cavity which separates the tissue to be treated from surrounding tissue. Once spreading device is in appropriate position, two sterile optical fibers with two cannulae attached are inserted under ultrasound guidance through the stainless-steel tube into the cavity previously created, protruding from the distal end of the stainless-steel tube. Once the optical fibers are in place, saline solution is infused through one of the cannula attached to the optical fibers with the aid of an infusion pump. While filling the area to be treated, laser energy of 1470 nm and 980 nm are delivered in alternate sequence through each optical fiber. This laser wavelength combination allows higher amounts of tissue ablation due to the augmented heating effect as a consequence of increased laser energy absorption by the saline solution as well as reduced bleeding due to enhanced laser energy absorption by blood. The spreading device and the cavity created prevents or diminishes damaging the superficial fat layer while desired hyperthermia is achieved in the deep fat layers. During irradiation, remnant saline solution and generated fluids and solid debris are removed from the treatment site through the remaining cannula attached to the optical fibers with the aid of a suction pump. Next, an appropriate amount of filling gel containing growth factors, an anti-inflammatory drug and an analgesic is administered through the cannula previously used for infusing saline solution. Spreading device is removed through the remaining cannula attached to the optical fiber. With this method there is less risk of injuring the skin and the superficial fat layers, avoiding and/or diminishing undesired lumps or irregularities in the skin.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A method of internal tissue removal based on laser energy comprising the steps of introducing a fiber optic based laser energy delivery device through a newly created passage into the inside of body tissue to be treated; introducing a penetrating (infusing) fluid to said tissue; applying sufficient quantities of said laser energy to remove substantial amounts of unwanted tissue in the form of fluid (liquid, vapor), solid debris and/or a combination of these; and removing debris and penetrating fluid from said inside of treated tissue.

2. A method of internal tissue removal based on laser energy comprising the steps of:
a. creating an initial cavity at a tissue treatment site;
b. introducing an infusing fluid whose absorption overlapped at least one wavelength output of the laser energy;
c. irradiating said tissue treatment site with adequate laser energy to remove desired amount of unwanted tissue in the form of liquid, gas, vapor, solid debris and combination of these; and
d. removing debris and remnant infusing fluid from said treatment site during and after irradiation.

3. The method of internal tissue removal based on laser energy according to claim 2, further comprising an additional step of introducing healing or sealing substances inside said cavity created, to promote filling, healing or sealing of the treatment site.

4. The method of internal tissue removal based on laser energy according to claim 2 further comprising the step of introducing at least one waveguide into said initial cavity.

5. The method of internal tissue removal based on laser energy according to claim 3, wherein said filling, healing or sealing substance is selected from a group consisting of growing factors, autografts, allografts, isografts, scaffolds, modified autografts, grafts including stem cells, materials supporting the sealing, filling or healing after tissue removal, and combination of those.

6. The method of internal tissue removal based on laser energy according to claim 3, wherein said filling, healing or sealing substance further comprises a combination of analgesics and anti-inflammatory drugs to enhance filling, healing or sealing processes.

7. The method of internal tissue removal based on laser energy according to claim 3, wherein said filling, healing or sealing substance has a form selected from the group consisting of liquid form, solid form, gel form and a combination of these.

8. The use of the method according to claim 2 to treat benign prostate enlargement in benign prostate hyperplasia (BPH); necrotic tissue; damaged tissue; infected tissue; unhealthy tissue; tumorous tissue; and unwanted adipose tissue.

9. The method of internal tissue removal based on laser energy according to claim 1, further comprising an additional step of introducing healing or sealing substances inside said cavity created, to promote filling, healing or sealing of the treatment site.

10. The method of internal tissue removal based on laser energy according to claim 9, wherein said filling, healing or sealing substance is selected from a group consisting of growing factors, autografts, allografts, isografts, scaffolds, modified autografts, grafts including stem cells, materials supporting the sealing, filling or healing after tissue removal, and combination of those.

11. The method of internal tissue removal based on laser energy according to claim 9, wherein said filling, healing or sealing substance further comprises a combination of analgesics and anti-inflammatory drugs to enhance filling, healing or sealing processes.

12. The method of internal tissue removal based on laser energy according to claim 9, wherein said filling, healing or sealing substance has a form selected from the group consisting of liquid form, solid form, gel form and combination of those.

13. The use of the method according to claim 1 to treat benign prostate enlargement in benign prostate hyperplasia (BPH); necrotic tissue; damaged tissue; infected tissue; unhealthy tissue: tumorous tissue; and unwanted adipose tissue.

14. A treatment system for the enhanced removal of internal tissue comprising:
a) a laser energy source;
b) spreading means to open an initial cavity in tissue to be treated;
c) at least one waveguide to transmit laser energy into a tissue treatment site;
d) means to introduce an infusing fluid to said treatment site;
e) an infusing fluid which absorbs a wavelength emitted by said laser energy source; and
g) means to remove debris and remnant infusing fluid during and/or after radiation treatment.

15. The treatment system according to claim 14, wherein said waveguide is a fiber optic based laser energy delivery device.

16. The treatment system according to claim 14, wherein said means to introduce an infusing fluid to said treatment site is an infusion pump.

17. The treatment system according to claim 14, further comprising means to place and verify position of distal ends of said spreading device and said at least one waveguide at the start and during the treatment.

18. The treatment system according to claim 17, wherein said means comprises ultrasound devices, open magnetic resonance imaging devices (MRI) and image scopes.

19. The treatment system according to claim 14, wherein said spreading means is comprised of a shape-memory material, compressible for introduction, which expands to create an initial cavity for said at least one waveguide and said infusing fluid to enter.

20. The treatment system according to claim 14, wherein said means to remove unwanted tissue in the form of liquid, vapor, solid debris and combination of those is a suction pump.

21. The treatment system according to claim 14, wherein said laser energy source delivers one or more laser wavelengths according to the absorption properties of said infusing fluid.

22. The treatment system according to claim 14, wherein said laser energy source delivers one or more laser wavelengths preselected from a range of 980 to 1940 nm.

23. The treatment system according to claim 14, wherein said infusing fluid is selected form the group consisting of an aqueous isotonic, hypertonic or hypotonic solution that can absorb laser energy.

24. A treatment system for the enhanced removal of internal tissue comprising:
a laser energy source;
an introducing means for optical fiber;
multiple optical fibers to transmit laser energy into a tissue treatment site;
means to introduce a penetrating fluid to said treatment site;
an penetrating fluid which absorbs a wavelength emitted by said laser energy source; and
means to remove debris and remnant penetrating fluid during and/or after radiation treatment.

25. The treatment system according to claim 24, further comprising means to place and verify position of distal ends of said spreading device and said at least one optical fiber at the start and during the treatment.

26. The treatment system according to claim 24, wherein said means to remove debris and remnant penetrating fluid is a suction pump.

27. The treatment system according to claim 24, wherein said means to introduce a penetrating fluid to said treatment site is an infusion pump.
